(19) United States
(12) Patent Application Publication
(10) Pub. No.: US 2023/0265062 A1
(43) Pub. Date: Aug. 24, 2023

(54) 4-FURANAMIDES AND METHOD FOR THE PRODUCTION THEREOF

(71) Applicant: Bayer Aktiengesellschaft, Leverkusen (DE)

(72) Inventors: Guenter HOEMBERGER, Eppstein (DE); Andreas REMBIAK, Langerwehe (DE)

(21) Appl. No.: 18/005,226

(22) PCT Filed: Jul. 2, 2021
(86) PCT No.: PCT/EP2021/068287

§ 371 Date: Jan. 12, 2023

(30) Foreign Application Priority Data
Jul. 16, 2020 (EP) 20186138.2

(51) Int. Cl. C07D 307/56

(52) U.S. Cl. CPC C07D 307/56

(57) ABSTRACT

The present invention relates to 4-furanamides of the general formula (I) and to a method for preparing said compounds and to the use thereof as important precursors for the synthesis of agrochemical and pharmaceutical active ingredients.

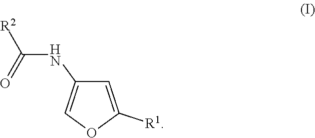

The present invention relates to 4-furanamides of the general formula (I) and to a method for preparing said compounds and to the use thereof as important precursors for the synthesis of agrochemical and pharmaceutical active ingredients.

4-Furanamides of the general formula (I) (especially R¹=COOMe) are important precursors of agrochemical (cf. WO2018/228985) and pharmaceutical active ingredients (e.g. Craig et al. Bioorganic & Medicinal Chemistry Letters, 12(18), 2647-2650; 2002).

4-Furanamides of the general formula (I) serve as starting material for the preparation of tetrahydro- and dihydrofurancarboxylic acids and esters. F. Brucoli, et al. Bioorganic & Medicinal Chemistry, 20(6), 2019-2024; 2012 describes a synthesis of Boc-protected 4-aminofuran.

The compounds of the general formula (I) are currently unknown but may serve as important building blocks for the synthesis of agrochemical and pharmaceutical active ingredients. Accordingly, the synthesis of compounds of the general formula (I) have not been described to date. The analogue synthesis of F. Brucoli et al. has the disadvantage that it has a long reaction time and a relatively low yield.

Sperotto et. al. Dalton Trans. 2010, 39, 10338-10351 reports the addition of copper powder in the Ullmann reaction in order to suppress side reactions and to optimize the yield of the desired reaction. This results in the fact that the method—at least for industrial-scale application—is not environmentally friendly.

Ji et al. J. Org. Chem. 2012, 77 7471 reports the function of ascorbic acid in Cu(I) catalyzed amination, namely as ligand and reducing agent.

Wang et al. (Org. Process Res. Dev. 2019, 23, 1918) describe a Cu(I) catalyzed amidation with addition of sodium ascorbate, but only for the coupling of phenyl-substituted, i.e. simple aromatic systems.

The disadvantages described in Brucoli et al. and Sperotto et al. render the method for preparing compounds of the formula (I) expensive, uneconomic and not very environmentally friendly.

In the light of the prior art described above, the object of the present invention is to find a method for preparing the compounds specified, such that the compounds of the general formula (I) can be obtained at higher yield, in higher purity and in an environmentally friendly manner, so that important intermediates for the preparation of active ingredients can be obtained on an industrial scale.

The object described above is achieved by a method for preparing compounds of the general formula (I)

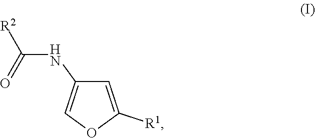

in which
R¹ is COO(C₁-C₄)-alkyl,
R² is CF₃, CF₂H, C₂F₅, CF₂C₁, CCl₃,
characterized in that the compounds of the general formula (II)

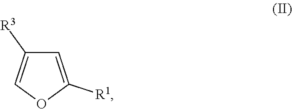

in which
R³ is halogen and
R¹ has the definitions stated above,
are reacted in the presence of compounds of the general formula (III) R²CONH₂, a Cu(I) salt, an amine, a base and ascorbic acid.

Preferred definitions of the radicals of the compounds of the general formulae (I), (II) and (III) are as follows:
R¹ is COOCH₃, COOC₂H₅,
R² is CF₃, CF₂H,
R³ is Br, Cl.

Particularly preferred definitions of the radicals of the compounds of the general formulae (I), (II) and (III) are as follows:
R¹ is COOCH₃,
R² is CF₃,
R³ is Br.

The present invention further relates to compounds of the general formula (I)

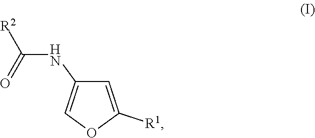

in which
R¹ is COO(C₁-C₄)-alkyl,
R² is CF₃, CF₂H, C₂F₅, CF₂Cl, CCl₃.

Preferred definitions of the radicals of the compounds of the general formula (I):
R¹ is COOCH₃, COOC₂H₅,
R² is CF₃, CF₂H.

Particularly preferred definitions of the radicals of the compounds of the general formula (I):
R¹ is COOCH₃,
R² is CF₃.

The reaction conditions for preparing compounds of the formula (I) are shown in Scheme 1.

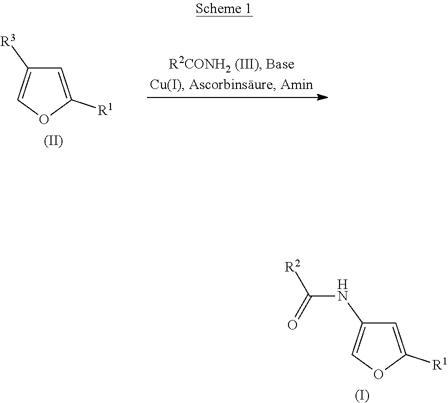

The compounds of the formula (II) react with compounds of the general formula (III), a Cu(I) salt, an amine and a base—optionally in the presence of a suitable solvent—to give compounds of the general formula (I).

Suitable bases are alkali metal carbonates or trialkali metal phosphates. Preference is given to using potassium carbonate as base.

A copper(I) salt is used, preference being given to copper(I) halide such as CuI or CuBr. Particular preference is given to CuBr.

The molar ratio of the compound of the general formula (II) to compounds of the general formula (III) is in the range of about 1:10 to 1:1, preferably from 1:2 to 1:1, especially preferably from 1:1.5 to 1:1.1.

The reaction is usually carried out in a temperature range of 70-100° C. The reaction is preferably carried out at 80-90° C., especially preferably at 83-85° C.

The reaction is usually carried out in a solvent, dioxane being preferred.

The precise mechanism of this Buchwald type Ullmann coupling is not yet clearly explained. It is known, however, that in the case of similar reactions, for example the addition of copper powder to the copper(I) catalyst, the reaction may be accelerated (Sperotto et al. Dalton Trans. 2010, 39, 10338-10351), presumably by suppressing the detrimental disproportionation of Cu(I), which is necessary according to the SET(single electron transfer) mechanism.

It has now been found, surprisingly, that ascorbic acid (vitamin C) is able to accelerate the reaction according to the invention, i.e. that even heteroaromatic systems (aromatic systems S. Wang et al) can be subjected to a Cu(I) catalyzed amidation using slightly different reaction conditions than in the case of Wang et al.

The advantage of vitamin C is that, compared to Sperotto et al., no additional copper is required, whereby the method is more environmentally friendly.

### DESCRIPTION OF THE METHODS AND INTERMEDIATES

### EXAMPLES

The present invention is elucidated in more detail by the examples which follow, without restricting the invention to these examples.

### Method of Measurement

The products were characterized by ¹H-NMR.

### Example 1

### Methyl 4-[(2,2,2-trifluoroacetyl)amino]furan-2-carboxylate

200 ml of dry 1,4-dioxane, a solution of 100 g (0.48 mol) of methyl 4-bromofuran-2-carboxylate (99%) in 100 ml of dry 1,4-dioxane and a solution of 63.2 g (0.53 mol) of trifluoroacetamide (95%) in 100 ml of dry 1,4-dioxane are initially charged in a dry 1000 ml double jacket vessel, equipped with a mechanical stirrer, at 22° C. under nitrogen. To this are added 7.1 g (0.048 mol) of copper(I) bromide (98%), 6.4 g (0.036 mol) of ascorbic acid (99%) and 134.8 g (0.96 mol) of finely powdered potassium carbonate (99%). The inlet opening is rinsed with 90 ml of dry 1,4-dioxane. The vessel is sealed and nitrogen is passed through the stirred suspension for 15 minutes. The reaction mixture is heated to 83-85° C. On reaching a temperature of ca. 70° C., a solution of 8.5 g (0.058 mol) of trans-N,N′-dimethylcyclohexane-1,2-diamine (97%) in 8.5 g of dry 1,4-dioxane are added. The mixture is then stirred at 83-85° C. for 3 hours. The mixture is then cooled to 15° C. and added in portions to a mixture of 250 ml of ethyl acetate and 560 g of hydrochloric acid (10%) cooled to 10° C. The reaction vessel is rinsed with 100 ml of ethyl acetate and 40 ml of hydrochloric acid (10%). The biphasic mixture is stirred at 20° C. for 30 minutes, then the phases are separated. The aqueous phase is re-extracted with 150 ml of ethyl acetate. The combined organic phases are washed twice with 300 ml each time of hydrochloric acid (1%). At a jacket temperature of 50° C. and at 200 mbar, ca. 500 ml of solvent are distilled off from the organic phase. Then, 250 ml of toluene are added, whereupon product may precipitate. Ca. 150 ml of solvent are distilled off at 50° C. jacket temperature and down to 115 mbar pressure. 50 ml of methanol are added to the residue, whereupon a clear solution forms at 50° C. Ca. 100 ml of solvent are distilled off therefrom at 50° C. and 115 mbar, whereupon product crystallizes out. Finally, the mass of the residue is ca. 200 g. The residue is cooled to 0° C. over 2 hours and stirred for 1 hour. The solid is filtered off and washed with 3 100 ml portions of cold toluene. The moist product is dried at 40° C. and 10 mbar.

This gives 72 g of methyl 4-[(2,2,2-trifluoroacetyl)amino]furan-2-carboxylate (99%) in 62% yield.

¹H-NMR (600 MHz, DMSO): δ 3.85 (s, 3H), δ 7.30 (s, 1H), δ 8.32 (s, 1H), δ 11.80 (br s, 1H)

¹³C-NMR (600 MHz, DMSO): δ 52.05 (s), δ 111.89 (s), δ 112.79, 114.69, 116.59, 118.5 (qa), δ 124.60 (s), δ 137.21 (s), δ 142.40 (s), δ 153.54, 153.79, 154.04, 154.29 (qa), δ 158.04 (s).

1. A method for preparing a compound of formula (I) 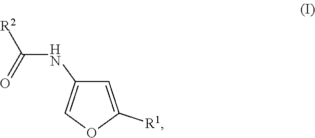
in which
R¹ is COO(C₁-C₄)-alkyl,
R² is CF₃, CF₂H, C₂F₅, CF₂Cl, CCl₃,
comprising reacting the compound of formula (II) 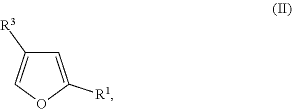
in which
R³ is halogen and
R¹ has the definitions stated above,
in the presence of a compound of formula (III) R²CONH₂, a Cu(I) salt, an amine, a base and ascorbic acid.

2. The method according to claim 1, wherein the definitions of the radicals of the compound of formulae (I), (II) and (III) are as follows:
R¹ is COOCH₃, COOC₂H₅,
R² is CF₃, CF₂H,
R³ is Br, Cl.

3. The method according to claim 1, wherein the definitions of the radicals of the compound of formulae (I), (II) and (III) are as follows:
R¹ is COOCH₃,
R² is CF₃,
R³ is Br.

4. The method according to claim 1, wherein the reaction is carried out at 80-90° C.

5. The method according to claim 1, wherein the solvent is dioxane.

6. The method according to claim 1, wherein the copper salt is Cu Br.
